(19) United States
(12) Patent Application Publication
(10) Pub. No.: US 2005/0171165 A1
(43) Pub. Date: Aug. 4, 2005

(54) Process for the preparation of perindopril

(76) Inventors: Shekhar Bhaskar Bhirud, Navi Mumbai (IN); Suhail Ahmed, New Delhi (IN); Batchu Chandrasekhar, Navi Mumbia (IN); Vandanapu Loka Appala Purushotham, Navi Mumbai (IN)

Correspondence Address: DILWORTH & BARRESE, LLP, 333 EARLE OVINGTON BLVD., UNIONDALE, NY, 11553, US

(21) Appl. No.: 10/985,097

(22) Filed: Nov. 10, 2004

Related U.S. Application Data
(60) Provisional application No. 60/569,041, filed on May 7, 2004.

(30) Foreign Application Priority Data
Nov. 12, 2003 (IN) 1179/MUM/2003

(51) Int. Cl. C07D 291/02; A61K 31/433

(52) U.S. Cl. 514/360; 548/122

(57) ABSTRACT

A process for preparing a novel intermediate in the preparation of perindopril is provided. Also provided are improved processes for the preparation of perindopril erbumine comprising (a) reacting a compound of Formula (15) 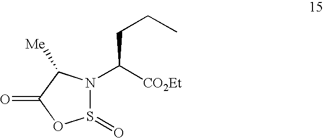
with a silylated octahydroindole-1H-2-carboxylic acid to form perindopril; and (b) reacting perindopril with tert-butylamine to form perindopril erbumine of Formula I:

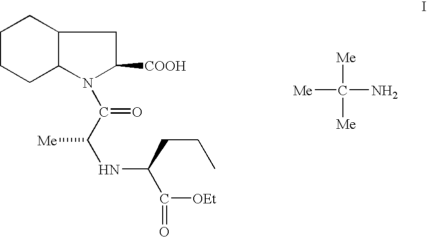

### PRIORITY

This application claims priority under 35 U.S.C. §119 to U.S. Provisional Application No. 60/569,041, filed May 7, 2004, and from Indian Provisional Application No. 1179/Mum/2003, filed Nov. 12, 2003, the contents of which are incorporated by reference herein.

### BACKGROUND OF THE INVENTION

1. Technical Field

The present invention generally relates to an improved process for the preparation of perindopril. More specifically, the present invention relates to a process which forms a novel intermediate in the preparation of perindopril.

2. Description of the Related Art

The present invention relates to a process for the preparation of perindopril erbumine (also known as (2S,3 μS,7 μS)-1-[(S)-N-[(S)-1-carboxy-butyl]alanyl]hexahydro-2-indolinecarboxylic acid, 1-ethyl ester, compound with tert-butylamine (1:1)) of Formula I: 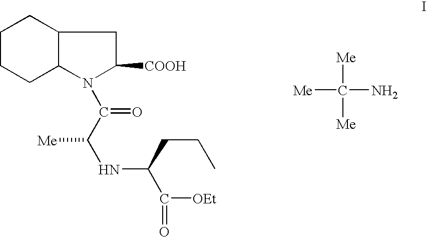
The tert-butylamine salt of perindopril, also known as perindopril erbumine, is the form commercially sold under the trade name Aceon®. Perindopril is the free acid form of perindopril erbumine and is an ethyl ester of a non-sulfhydryl angiotensin-converting enzyme (ACE) inhibitor. Perindopril is a pro-drug and is metabolized in vivo by hydrolysis of the ester group to form perindoprilat, the biologically active metabolite. Perindopril is ordinarily used to treat hypertension.

It is believed that perindopril lowers blood pressure primarily through inhibition of ACE activity. ACE is a peptidyl dipeptidase that catalyzes conversion of the inactive decapeptide, angiotensin I, to the vasoconstrictor, angiotensin II. Angiotensin II is a potent peripheral vasoconstrictor, which stimulates aldosterone secretion by the adrenal cortex, and provides negative feedback on renin secretion. Inhibition of ACE results in decreased plasma angiotensin II, leading to decreased vasoconstriction, increased plasma renin activity and decreased aldosterone secretion. The latter results in diuresis and natriuresis and may be associated with a small increase of serum potassium.

U.S. Pat. No. 4,914,214, incorporated by reference herein, discloses processes for the preparation of perindopril. In one process for the preparation of perindopril, indoline-2-carboxylic acid (1) is hydrogenated in methanol over a rhodium-aluminum oxide (Rh/Al₂O₃) catalyst to form (2S,3aS,7aS)-octahydroindole-2-carboxylic acid of the formula (2). The acid of formula (2) is esterified with thionyl chloride and benzyl alcohol to yield (2S,3aS,7aS)-octahydroindole-2-carboxylic acid benzyl ester (3), which is one key intermediate of perindopril. Another key intermediate of perindopril is prepared by reacting L-norvaline (4) with thionyl chloride and ethanol to form an ethyl ester (5). The ethyl ester (5) is reacted with sodium pyruvate (6) and subjected to hydrogenation to form N-1S-carboxyethylbutyl-(S)-alanine (7), another key intermediate. (2S,3aS,7aS)-octahydroindole-2-carboxylic acid benzyl ester (3) is then coupled with N-1S-carboxyethylbutyl-(S)-alanine (7) in presence of sodium dicyclohexyl dicarbodiimide (DCC) to yield perindopril benzylated ester (8). Perindopril benzylated ester (8) is hydrolyzed to form perindopril (9). Perindopril (9) is reacted with tert-butylamine to form the perindopril erbumine salt (I) as shown in Scheme I:

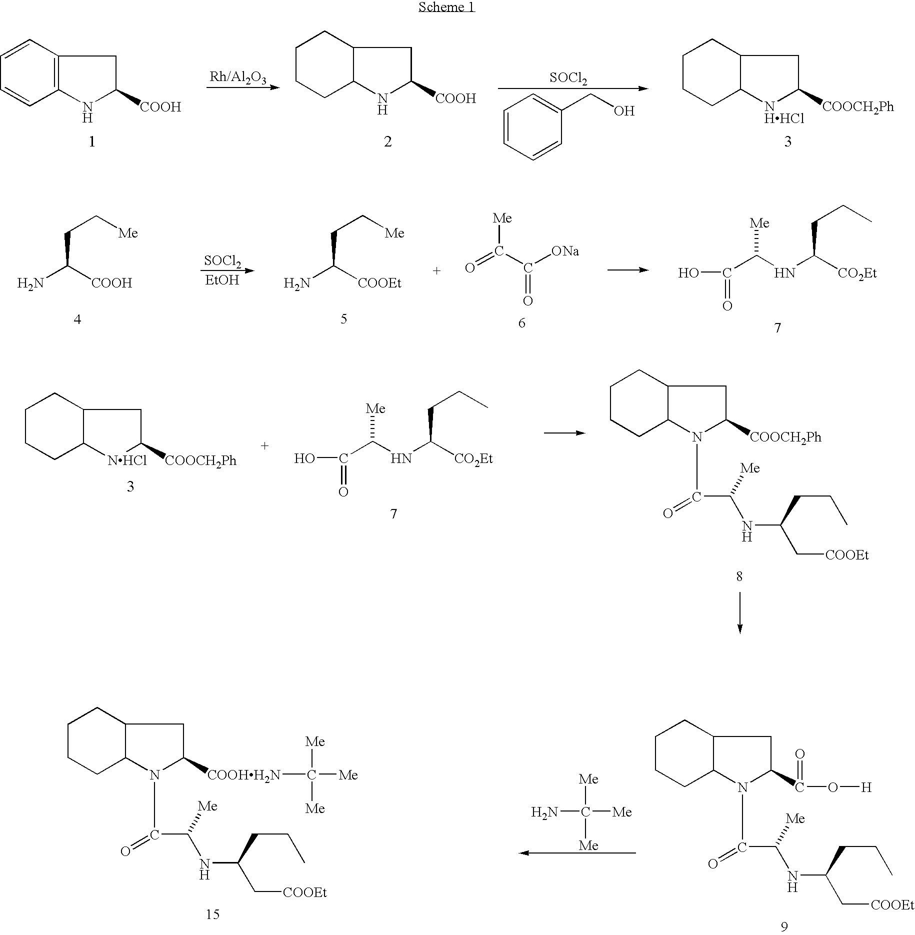

European Patent Application EP 1279665 to Cid, incorporated by reference herein, also discloses a process for the preparation of perindopril. In this process, an N-carboxyanhydride (NCA) is formed in-situ. The intermediate N-1S-carboxyethyl butyl-(S)-alanine (7) is reacted with carbonyl diimidazole (10) to form the N-carboxyanhydride (11) as shown in Scheme II: 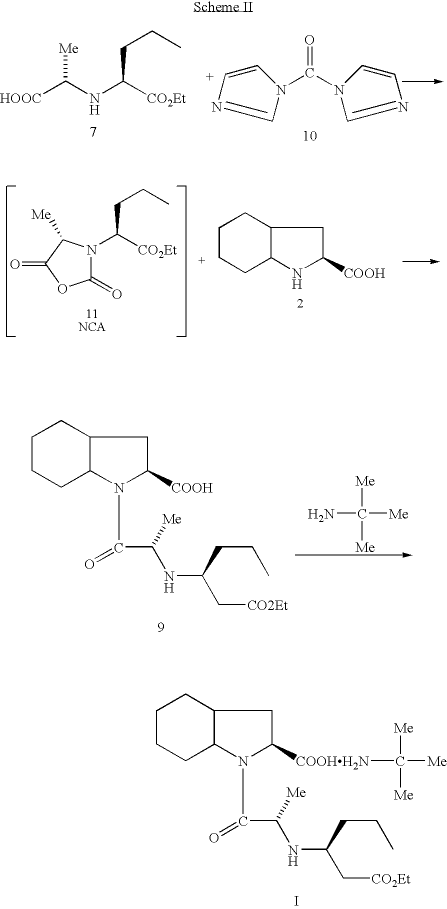
N-carboxyanhydride (11) is then reacted with (2S,3aS,7aS)-octahydroindole-2-carboxylic acid (2) to yield perindopril (9). Again, the perindopril (9) is reacted with t-butylamine for the perindopril erbumine salt (I).

Several drawbacks are associated with these processes. These drawbacks include the use of toxic reagents with stringent standards, expensive reagents that are not recyclable, and numerous steps that add to process inefficiencies and complications during commercial production.

Accordingly, there remains a need for improved processes for preparing perindopril that eliminates and reduces the drawbacks of the prior art in a convenient and cost efficient manner on a commercial scale.

### SUMMARY OF THE INVENTION

In one embodiment of the present invention, a process for the preparation of an intermediate of perindopril is provided. The intermediate N-sulfoxyanhydride (NSA) of Formula 15: 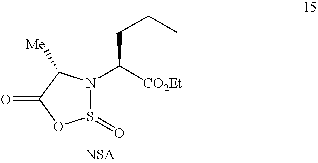
is also provided and may be prepared by reacting N-(1S)-carboxyethoxybutyl-(S)-alanine with chlorosulfinyl imidazole optionally in the presence of a solvent.

In another embodiment of the present invention, a process for preparing perindopril erbumine is provided comprising:
(a) reacting N-(1S)-carboxyethoxybutyl-(S)-alanine with chlorosulfinyl imidazole optionally in the presence of a solvent to form NSA; and,
(b) converting the NSA to perindopril.

In accordance with yet another embodiment of the present invention, a process for preparing perindopril erbumine is provided comprising:
(a) reacting NSA with a silylated octahydroindole-1H-2-carboxylic acid to form perindopril; and
b) reacting the perindopril with tert-butylamine to form perindopril erbumine.

The advantages of the present invention include at least the following:
1) The use of NSA as a starting substance provides an effective industrial process for preparation of perindopril erbumine because the condensation reaction may be performed at lower temperatures, which is more favorable to industrial scale processes.
2) The use of NSA as a starting substance also provides an economical benefit because solvents typically used in the preparation of NSA may be isolated quantitatively and recycled and some reactants may be regenerated and recycled.

### DETAILED DESCRIPTION OF THE PREFERRED EMBODIMENTS

In one aspect of the present invention, an N-sulfoxyanhydride (15) is formed in-situ by reaction of N-1S-carboxyethylbutyl-(S)-alanine (7) with chlorosulfinyl imidazole (14) as shown in Scheme III:

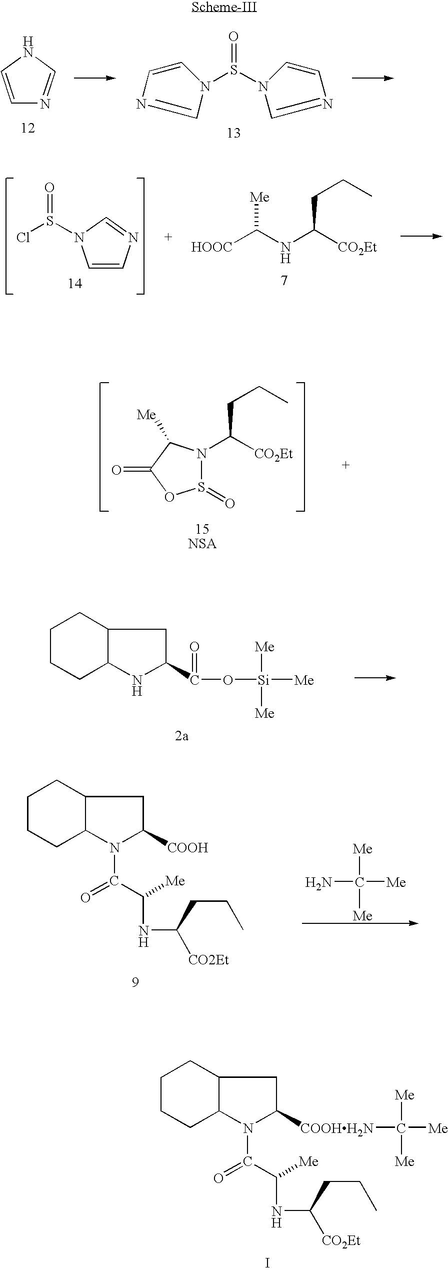

The preparation of chlorosulfinyl imidazole (14) is disclosed in, for example, Ogata Masaru and Matsumoto Hiroshi, “N(Chlorosulfinyl)-imidazole as a new imidazole transfer reagent” Synthetic Communications, 10(10), pp. 733-742 (1980), and Coll, Alberto Palomo, “Formation of new N-sulfoxyanhydrides as intermediates for the synthesis of ACE inhibitors” Afinidad 57(487), pp. 209-210 (2000), the contents of which are incorporated by reference herein. Generally, chlorosulfinyl imidazole (14) may be prepared by reacting imidazole (12) with SOCl₂ in an about 1:4 ratio in dry methylene chloride yielding N,N′-thionyldiimidazole hydrochloride (13). N,N′-thionyldiimidazole hydrochloride (13) can then be filtered off and an equimolar amount of thionyl chloride is added to the filtrate to yield chlorosulfinyl imidazole (14).

In accordance with the present invention, chlorosulfinyl imidazole (14) is reacted with N-1S-carboxyethylbutyl-(S)-alanine (7) to form NSA (15). The reaction can be carried out at a temperature ranging from about −15° C. to about 25° C. for a time period ranging from about 60 minutes to about 90 minutes. The molar ratio of chlorosulfinyl imidazole to N-1S-carboxyethylbutyl-(S)-alanine can vary widely, e.g., a molar ratio of chlorosulfinyl imidazole to N-1S-carboxyethylbutyl-(S)-alanine ranging from about 1:1.1 to about 1:1.2.

If desired, this reaction can take place in the presence of substantially dry organic solvents (e.g., solvents having a moisture content of less than about 0.04%). Suitable dry organic solvents include, but are not limited to, chlorinated organic solvents, non-chlorinated solvents and the like and mixtures thereof. The chlorinated organic solvents may be, for example, methylene chloride. The non-chlorinated solvents may be, for example, ethyl acetate, dimethyl carbonate, diethyl carbonate, and acetonitrile. The solvent is generally added in an amount of from about 0 to about 20 wt. percent. The organic solvent may be regenerated and recycled. Any imidazole hydrochloride that is formed may be filtered off.

After performing the reaction to form NSA, the NSA (15) can be left in the organic solvent, if used. The presence of the NSA in the solvent may be confirmed by the quantitative isolation of the hydrochloride form of chlorosulfinyl imidazole in two steps: (1) iodometric titration of the SO₂ formed after hydrolysis of NSA (IR spectroscopy of the solution having characteristic absorption bands at 1820 cm⁻¹, 1750 cm⁻¹, and 1030 cm⁻¹); and (2) reacting the NSA with silylated amino acids under release of SO₂.

The NSA (15) is then reacted with a silylated (2S,3aS,7aS)-octahydoindole-2-carboxylic acid (2a) to form perindopril. This reaction may be performed at a pH ranging from about 2 to about 6. Alternatively, the reaction may be performed at a pH over 7 in the presence of inorganic or organic salts of N-1S-carboxyethylbutyl-(S)-alanine (7). In one embodiment of the present invention, the inorganic salt of N-1S-carboxyethylbutyl-(S)-alanine (7) is selected from the potassium salts or sodium salts or combinations thereof. In another embodiment of the present invention, the organic salt of N-1S-carboxyethylbutyl-(S)-alanine (7) is selected from the group consisting of 1,8-diazabicylo[5.4.0.]undec-7-ene (DBU), 1,5-diazabicyclo[4.3.0]non-5-ene (DBN), triethylamine (TEA), tetramethylguanidine, imidazole, and methylimidazole salts of N-1S-carboxyethylbutyl-(S)-alanine. The reaction between NSA (15) and silylated octahydoindole-2-carboxylic acid (2a) may also be performed in the presence of the free amino acid form of N-1S-carboxyethylbutyl-(S)-alanine (7).

When the free amino acid form is used, N-1S-carboxyethylbutyl-(S)-alanine (7) is activated with N-chlorosulfinyl imidazole to form N-ethoxycarbonylbutylalanine-N-sulfoxyanhydride (15). This NSA is reacted with a silylated 2S,3aS,7aS octahydro-1H-indole-2-carboxylic acid (2) to form perindopril. During the reaction, gaseous SO₂ is released, which may also be used to detect the NSA. Generally, the reaction between NSA and silylated 2S,3aS,7aS octahydro-1H-indole-2-carboxylic acid may be carried out at a temperature ranging from about −20° C. to about 25° C. for a time period sufficient to form perindopril, e.g., a time period of no more than about 90 minutes. The molar ratio of NSA to silylated 2S,3aS,7aS octahydro-1H-indole-2-carboxylic acid can range from about 1:0.9 to about 1:1.1.

The perindopril is further reacted with tert-butylamine as known in the art to form perindopril erbumine. The reaction of perindopril and tert-butylamine may be performed in ethyl acetate at a temperature ranging from about 25° C. to about 35° C.

The following examples are provided to enable one skilled in the art to practice the invention and are merely illustrative of the invention. The examples should not be read as limiting the scope of the invention as defined in the claims.

### EXAMPLE 1

Step I: Preparation of silylated (2S,3aS,7aS)-octahydro-1H-indole-2-carboxylic acid

Trimethyl chlorosilane (10.86 g, 0.1M) was added to a mixture of octahydroindole-1H-2-carboxylic acid (15.2 g, 0.09M), anhydrous methylene chloride (300 ml) and triethylamine (10.1 g, 0.1M) and the mixture was stirred for 2 hours until silylated octahydro-1H-indole-2-carboxylic acid was formed.

Step II: Preparation of NSA

Thionyl chloride (14.28 g, 0.120M) was added to dry methylene chloride (150 ml) and the mixture was cooled to a temperature of about −5° C. Imidazole (16 g, 0.22) was then added and the mixture was stirred for about 65 minutes at a temperature ranging from about −5° C. to about 0° C. N-1(S)-carboxyethylbutyl-(S)-alamine (19.95 g, 0.1M) in methylene chloride (200 ml) was added. The mixture was stirred for about 65 minutes while increasing the temperature to a range of from about 20° C. to about 25° C. until NSA was formed.

Step III: Preparation of Perindopril Erbumine

The silylated (2S,3aS,7aS)-octahydro-1H-indole-2-carboxylic acid in methylene chloride from Step I was cooled to a temperature of about −15° C. and was added to the reaction mixture containing NSA from Step II under stirring. The reaction was maintained for a few hours at a temperature of about 25-35° C. A slightly yellow solution was formed and was evaporated on a Rota vapor. The solvent was removed and water (100 ml), sodium chloride 25 g and ethyl acetate (200 ml) were added to the residue. The pH was from about 2.5 to about 3.0 and was adjusted with a 10% NaOH solution to a pH ranging from about 4.2 to about 4.5. The organic phase was separated and the aqueous phase was extracted with ethyl acetate (100 ml×2). The combined ethyl acetate phases were dried with anhydrous sodium sulphate (50 g). The dried organic layer was concentrated on a rotavapor bath at 40° C. under vacuum to get an oily residue. (Weight: 30 g, %-yield: 90%, purity: 92 to 94% by HPLC).

The oily residue was dissolved in 300 ml ethyl acetate and tert-butylamine (6 g, 0.08 mol) was added at room temperature under stirring for about 60 minutes to about 90 minutes. The precipitated product was filtered and further recrystallized in ethyl acetate (350 ml) to get perindopril erbumine. (Weight: 28 g, %-yield: 65%; purity: ˜99.5%) Specific optical rotation [α]n=−66 (C=1%, MeOH), IR (KBr) spectrum shows the following absorptions cm−1 3300, 2930, 1744, 1732 m 1644 m 1568. The 1H-NMR (CDCl₃) shows the following signals at δ 4.28-4.12 (m, 1H), 4.18-4.09 (q, 2H), 3.76 (m, 2H) 3.53 (q, 1H), 3.1 (t, 1H), 2.32-2.14 (m, 2H), 2.01 (m, 1H), 1.75-1.62 (m, 4H), 1.32 (m, 2H), 1.30 (S, 9H), 1.28 (t, 3H), 0.88 (t, 3H). CI Mass shows m/z at 368 (M+).

It will be understood that various modifications may be made to the embodiments disclosed herein. Therefore the above description should not be construed as limiting, but merely as exemplifications of preferred embodiments. For example, the functions described above and implemented as the best mode for operating the present invention are for illustration purposes only. Other arrangements and methods may be implemented by those skilled in the art without departing from the scope and spirit of this invention. Moreover, those skilled in the art will envision other modifications within the scope and spirit of the claims appended hereto.

1. A compound of Formula 15:

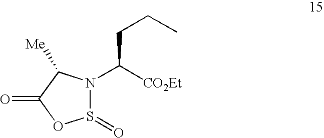

2. A process for the preparation of a compound of Formula 15 (NSA): 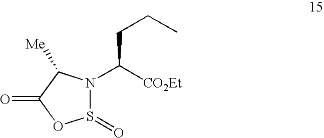
comprising reacting chlorsulfinyl imidazole with N-(1S)-carboxyethylbutyl-(S)-alanine.

3. The process of claim 2, wherein the reaction is carried out in a solvent.

4. The process of claim 3, wherein the solvent is selected from the group consisting of chlorinated organic solvents, non-chlorinated organic solvents and combinations thereof.

5. The process of claim 4, wherein the chlorinated organic solvent is methylene chloride.

6. The process of claim 4, wherein the non-chlorinated organic solvent is selected from the group consisting of ethyl acetate, dimethyl carbonate, diethyl carbonate, acetonitrile and combinations thereof.

7. The process of claim 3, wherein the moisture content of the solvent is less than about 0.04%.

8. The process of claim 2, wherein the reaction is carried out at a temperature of about −15° C. to about 25° C.

9. The process of claim 2, wherein the molar ratio of the chlorosulfinyl imidazole to the N-1S-carboxyethylbutyl-(S)-alanine is about 1:1.1 to about 1:1.2.

10. The process of claim 2, wherein NSA is thereafter converted to perindopril.

11. A process for preparing perindopril erbumine of the Formula I: 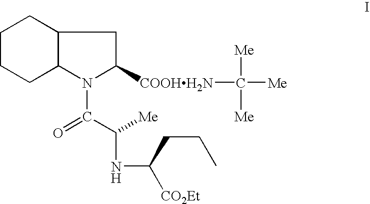
comprising (a) reacting a compound of Formula 15 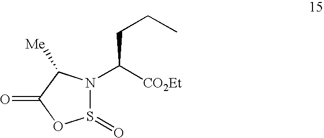
with a silylated octahydroindole-1H-2-carboxylic acid to form perindopril; and,
(b) reacting the perindopril with tert-butylamine to form perindopril erbumine of Formula I.

12. The process of claim 1 wherein the reaction of the compound of Formula 15 and silylated octahydroindole-1H-2-carboxylic acid is carried out in the presence of an organic salt of N-1S-carboxyethylbutyl-(S)-alanine, an inorganic salt of N-1S-carboxyethylbutyl-(S)-alanine and combinations thereof.

13. The process of claim 12, wherein the inorganic salt of N-1S-carboxyethylbutyl-(S)-alanine is selected from the group consisting of potassium salts, sodium salts and combinations thereof.

14. The process of claim 12, wherein the organic salts of N-1S-carboxyethylbutyl-(S)-alanine (7) are selected from the group consisting of 1,8-diazabicylo[5.4.0.]undec-7-ene (DBU), 1,5-diazabicyclo[4.3.0]non-5-ene (DBN), triethylamine (TEA), tetramethylguanidine, imidazole, and methylimidazole salts of N-1S-carboxyethylbutyl-(S)-alanine.

15. The process of claim 11, wherein the reaction in step (a) is carried out at a temperature of about −20° C. to about 25° C.

16. The process of claim 11, wherein the reaction in step (b) is carried out at a temperature of about 25° C. to about 35° C.

17. The process of claim 11, wherein the molar ratio of the compound of Formula 15 to the silylated 2S,3aS,7aS octahydro-1H-indole-2-carboxylic acid is about 1:0.9 to about 1:1.1.
